(19) United States
(12) Patent Application Publication
(10) Pub. No.: US 2011/0015384 A1
(43) Pub. Date: Jan. 20, 2011

(54) SULFURIZATION AGENT AND ITS USE

(75) Inventors: Peter Christian De Visser, Leiden (NL); Gerardus Johannes Platenburg, Voorschoten (NL)

Correspondence Address: YOUNG & THOMPSON, 209 Madison Street, Suite 500, Alexandria, VA, 22314, US

(73) Assignee: PROSENSA HOLDING BV, Leiden (NL)

(21) Appl. No.: 12/866,608

(22) PCT Filed: Feb. 6, 2009
(86) PCT No.: PCT/NL2009/050053

§ 371 Date: Aug. 6, 2010

(30) Foreign Application Priority Data
Feb. 8, 2008 (EP) 08151195.8

(51) Int. Cl. C07H 19/20; C07F 9/205

(52) U.S. Cl. 536/26.71; 558/122

(57) ABSTRACT

The use of a sulfurizing agent of formula A: or a salt, hydrate, solvate, or a mixture thereof, in which all R groups, independently, represent H or an organic group, in sulfurization. The sulfurizing agent is preferably a formamidine disulfide. It is found a particularly suitable alternative to existing sulfurizing agents, since it is easy to synthesize from readily available, cheap starting materials.

### FIELD OF THE INVENTION

The invention pertains to compounds for use as a sulfurization agent, i.e. to oxidize a compound, in particular a phosphorus-containing compound, by incorporating a double-bonded sulfur atom into said compound. These sulfurization agents are found e.g. particularly useful in stabilizing internucleotide linkages and creating flame retardants.

### BACKGROUND OF THE INVENTION

Oligonucleotides and their synthetic derivatives have become important assets as both research tools and pharmaceuticals. Their effects are versatile, from recruiting RNase H to digest target mRNA, via exon-skipping mediated therapeutics and antisense blockage of biological relevant startcodon targets to small interfering RNAs (siRNA) tools & therapeutics.

Stability of the nucleic acid is an important factor in the effectiveness of such tools and pharmaceuticals, as, for example, the regular phosphate linkages in DNA and RNA are subject to hydrolysis. To circumvent undesired processes, more stable analogues of the natural nucleic acids have been synthesized, including PNA, ENA/BNA, LNA and PMO. Besides, stabilization could be obtained by alkylation of the 2′-OH group in RNA, leading to a series of derivatives, among which 2′-O methylated and allylated derivatives. An additional stabilizing factor can be introduced by replacing the phosphate (P═O) internucleotide linkages with phosphorothioate (P═S).

Phosphorus derivatives of peptides are becoming increasingly important in chemical and biological research. Phosphorylated proteins, whereby a phosphate monoester is formed with the side-chain hydroxyl group of serine, threonine, or tyrosine, have been identified as key intermediates in protein regulation and signal transduction by protein kinases and phosphatases. Phosphorylated peptides (phosphopeptides, Curr. Org. Chem. 2007, 11, 409) are being employed to understand the mechanism of action of phosphorylation and the role of phosphorylation in disease.

Sulfurized (P═S containing) analogues of these phosphorus-containing moieties as envisioned in this invention will be important to the advancements in these fields. The introduction of P═S bonds can be accomplished by application of a sulfurization reagent, which oxidizes the intermediate trivalent phosphorus atom, resulting from coupling of a new nucleotide, to a pentavalent atom.

Because natural sulfur, S₈, reacts only slowly with trivalent phosphorus, a number of sulfurization reagents have been prepared and assayed in the past decades. One of these is the common Beaucage reagent (J. Org. Chem. 1990, 55, 4693). Besides the Beaucage reagent, other reagents have found their way into the laboratory, especially in the field of oligonucleotide synthesis. Amongst them are phenylacetyl disulfide (PADS, Nucleos. Nucleot. 1999, 18, 485), tetraethylthiuram disulfide (TETD, Tetrahedron Lett. 1991, 32, 3005), bis(O, O-diisopropoxyphosphinothioyl) disulfide (S-tetra, Tetrahedron Lett. 1993, 33, 5317) and 3-ethoxy-1,2,4-dithiazoline-5-one (EDITH, Nucleos. Nucleot. 1997, 16, 1585).

WO-2005/097817 gives an extensive list of reagents for oligonucleotide synthesis and purification. The preferred reagent is 3-amino 1,2,4-dithiazolidine-5-one.

Unfortunately, the preparation of sulfurization reagents in the art is often quite elaborate. U.S. Pat. No. 5,852,168 reports that the synthetic accessibility, solubility properties and stability of the widely spread Beaucage reagent are not optimal, and it questions the suitability of the same reagent for large-scale oligonucleotide preparation. A sulfurization compound that would be easy to synthesize from readily available, cheap starting materials would be a welcome replacement for the above reagents.

Zhiwei Wang et al. “Dimethylthiuram Disulfide. New Sulfur Transfer Reagent in Phosphorothioate Oligonucleotide Synthesis” Methods in Molecular Biology, Vol. 288 (2005), p. 51-63 compare dimethylthiuram disulfide (DTD) as a sulfurization agent with the aforementioned PADS. The authors conclude that DTD allows for an overall 20% reduction in solvent consumption and reduces the total synthesis time by 25%. U.S. Pat. No. 6,809,195 teaches similar disclosure. Their contents is herein incorporated by reference.

Unfortunately, as addressed at page 58 of Wang et al., DTD rapidly degrades upon addition of base. This disadvantageously restricts its applicability. Also, the synthesis involves inflammable and irritating ingredients, and DTD is accompanied with an obnoxious long-lasting smell.

Hence, the need for an easy-to-produce acid and base stable reagent continues to exist.

### SUMMARY OF THE INVENTION

It is an objective of the present invention to provide a sulfurization reagent that is not hindered by the aforementioned disadvantages, and is acid and base stable.

It is now found that a particular group of compounds, and especially formamidine disulfide (FMDS),

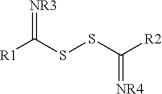

and its salts, fulfill these requirements. R₁-R₄ will be defined below. The stability of FMDS in alkaline conditions is demonstrated in the accompanying examples 1 and 2.

These compounds are well available in the art. Reaction of cheap thiourea and elemental halogen (e.g. Cl₂) under anhydrous conditions leads to quantitative FMDS dihalogenide formation (Tetrahedron 2005, 61, 4233), which can conveniently and straightforwardly replace any of the aforementioned reagents. However, up to present, no link is made between these compounds and their potential use in sulfurization.

It is noted that FMDS substantially differs from the DTD compounds subject of study in the above-cited paper by Wang et al. DTD synthesis as disclosed in Wang et al. does not yield tautomeric forms having SH moieties in any detectable amount, as revealed by NMR characterisation. This was beforehand unexpected based on the information given in Wang et al. itself, since the synthesis involves a concluding acid step, which prevents DTD from taking other tautomeric conformations. Moreover, Wang et al. make mention of a melting point rather than a melting range, thus indicating the absence of tautomeric impurities.

When compared to DTD, FMDS involves less synthesis steps and no disadvantageous smell at all.

Generalized, the scope of this invention not only extends to sulfurizing phosphorus-containing moieties, but also to the field of non-phosphorus organic materials (e.g. sulfurized olefins for lubrication, Chem. Technol. Fuels Oils 1986, 22, 570) as well as metal-containing compounds (e.g. catalysts (Fuel Process. Technol. 2004, 86, 169), anti-friction layers (Chem. Petrol. Engin. 1966, 2, 37), lubricants (Surf. Coat. Technol. 2000, 132, 1) and solar cells (Thin Solid Films 2001, 387, 80)), provided that the organic molecule can be brought to a higher oxidation state by attachment of a double-bonded sulfur atom.

### DETAILED DESCRIPTION OF THE INVENTION

The invention thus pertains to the use of sulfur-containing compounds having formula A:

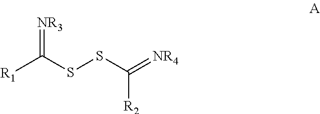

or a salt, hydrate, solvate, or a mixture thereof,
in which all R groups each independently represent H or (organic) groups, in sulfurization. Preferably, all R groups each independently represent a moiety selected from the group consisting of (substituted) amine, (substituted) hydroxyl, (substituted) sulfhydryl, (substituted) hydroxylamine, thiocyanate, isothiocyanate, cyanate, isocyanate, alkyl, alkenyl, alkynyl, aryl, aralkyl, all or not comprising one or more heteroatoms.

Alternatively or additionally, in formula A all R groups each independently have the meanings of H, —OH, —NO₂, —CN, —SO₂Rₐ, —SRₐ, —NHRₐ, —N(Rₐ)₂, —C(O)Rₐ, —CO₂Rₐ, —ORₐ, halogen, alkyl, alkenyl, alkynyl, aryl, aralkyl, alkoxyl, in sulfurization. Rₐ represents independently for each occurrence H, halogen, alkyl, aryl or aralkyl, optionally containing one or more heteroatoms, preferably selected from the group selected from O, N, P, Se, B and S.

The compounds of formula A are referred to herein as sulfur-transfer reagents or sulfurization reagents. In essence, the basis of these sulfurization reagents is formed by a reactive disulfide moiety, flanked on either side by a C═N bond. Either one, but preferably both C═N groups may be heterogeneously substituted alkyl, i.e. having R₃ and/or R₄ groups that are not H, and comprising one or more heteroatoms.

The terms “alkyl”, “alkenyl”, “alkynyl”, “aryl” and “aralkyl” comprise substituted and unsubstituted hydrocarbon radicals, preferably having from one to 20, more preferably 2-10 carbon atoms, and includes cyclic forms, such as cycloalkyl and cycloalkenyl.

“Aryl” preferably means an organic radical derived from an aromatic hydrocarbon containing 6 to 14 carbon atoms and includes monocyclic or condensed carbocyclic aromatic rings (e.g., phenyl, naphthyl, anthracenyl, phenanthrenyl, etc.) optionally further substituted with one to two substituents.

The above groups may contain one, two or even more substitutions, preferably selected from O, N, P, Se, B and S atoms. It is understood that if not specified otherwise, C, and the heterogeneous atoms present further comprise hydrogen atoms to properly satisfy the valency of the respective atom.

“(substituted) amine” means a NR₅R₆ group attached through the nitrogen atom, in which R₅ and R₆ can independently be H or an organic group as discussed above.

“(substituted) hydroxyl” means an OR_{S} group attached through the oxygen atom, in which R₇ can be H or an organic group as discussed above.

“(substituted) sulfhydryl” means an SR₈ group attached through the sulfur atom, in which R₈ can be H or an organic group, such as these discussed above.

In a preferred embodiment, R₁ and R₂ are nitrogen-containing moieties.

R₁ and R₂ groups can be an organic group involving multiple heteroatoms. For instance, R₁ and R₂ can, independently of one another and of the other R groups, comprise sulfur-containing radicals (—SZ, —SOZ, —SO₂Z), phosphorus-containing radicals (—PZ₂, —POZ₃, —PSZ₃), nitrogen-containing radicals (—NZ, —NZ₂, —NZ₃⁺), oxygen-containing radicals (—OZ), in which Z is amine, imine, oxygen, hydroxyl, sulfur, sulfhydryl, aryl, alkyl, alkenyl or alkynyl. In principle, Z may again comprise one or more heterogeneous atoms.

R₁ and R₂, and R₃ and R₄ may the same or different. Besides homogeneous compounds (i.e. R₁═R₂ and R₃═R₄), heterogeneous disulfides can also be applied in sulfurization (in which R₁≠R₂ and/or R₃≠R₄). In a preferred embodiment, groups R₃═R₄ and R₁═R₂. In a more preferred embodiment, R₃═R₄═H, and in a still more preferred embodiment, R₁═R₂═NH₂ yielding the compound known as formamidine disulfide.

In one embodiment, R₁ and R₂ is not SH. More preferably, R₁ and R₂ are not SH.

In another embodiment, the compound of structure is preferably represented by formula A1:

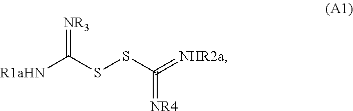

in which R₃ and R₄ have the above meanings, and R₁ₐ and R₂ₐ can each independently have the meaning as given to Rₐ above. In a more preferred embodiment, R₃═R₄═H, and in a still more preferred embodiment, R₁═R₂═NH₂ yielding the compound known as formamidine disulfide.

The compound of structure A (or A1) can be applied in its neutral form (as depicted) or as a salt, hydrate or solvate thereof. A preferred salt is an n HX salt, in which X is any halogen and n is 1-4. More preferably, the compound is in its dihydrochloride form.

### Synthesis

The synthesis of compounds of structure A, especially formamidine disulfide, is very simple, high-yielding, cost-effective and straightforward. As described by M. Soroka and W. Goldeman “Reinvestigation of the conversion of epoxides into halohydrins with elemental halogen catalysed by thiourea” (Tetrahedron 2005, 61, 4233), formamidine disulfide can be prepared from readily available thiourea through reaction with X₂ (where X=halogen, e.g. F, Cl, Br), leading quantitatively to the corresponding formamidine disulfide.2HX salt. It is therefore that the sulfurization reagent of structure A can be in the form of a salt, in general in its n HX salt wherein n=1-4 (1, 2, 3, 4). In a preferred embodiment, n is 2 and X═Cl.

The synthesis route for formamidine disulfide can be extrapolated straightforwardly to other compounds of formula A, suitable for sulfurizing e.g. organophosphites, that are the subject of the invention, without any undue experimentation.

### Application

The above-defined compound A is used in sulfurization. “Sulfurization” reactions involve oxidization of a particular atom to a higher oxidation state.

An important example of the above sulfurization is the oxidation of trivalent phosphorus P^{III} to pentavalent P^{V}, by means of attachment of a double-bonded sulfur atom to the atom at dispute.

Specifically, the present invention provides a method for the incorporation of phosphorotioate linkages (P═S) into a variety of molecules. In theory, the compound to be sulfurized can be any organic compound containing a trivalent phosphorus P^{III}, i.e. an organophospite. A phosphate or phosphonous ester can thus be sulfurized to its corresponding phosphorothioate or phosphonothionate, respectively. Compound A oxidizes the formed P^{III} center (in case of applying the phosphoramidite synthetic procedure, this will be a phosphotriester) to a pentavalent P═S containing intermediate.

An important class of organophosphate compounds is formed from biologically important molecules, such as DNA, RNA, and phosphopeptides, for example. Other phosphorus-containing materials of interest include phospholipids, phosphorus-containing polymers, and phosphonate-modified surfaces. Once oxidised to P^{V}, the most stable oxidation state for phosphorus, the organophosphate compounds cannot be oxidised further and are thus fire-resistant. Sulfurized organophosphates are important additives to confer fire-resistance to otherwise flammable materials such as wood, paper and textiles. Certain phosphorus polymers containing the thiophospho function (i.e., S═P(R)₃, where R is an alkyl group or heteroatom-containing alkyl group) are especially reported useful as flame retardants (see e.g. U.S. Pat. No. 5,852,168, its content hereby incorporated by reference herein). Hence, in one embodiment, phosphorus P^{III} is converted to a flame-retarding organophosphate compound having P^{V}. Surfaces modified with phosphonates have allowed for the preparation of metal-organic multilayers, similar to Langmuir-Blodget films. Such metal organic multilayers have many applications including chemo-selective and type-selective crystal growth and as chemoselective sensors.

Hence, in one aspect, the invention pertains to the sulfurization of a phosphite, phosphonite, phosphonamidite, phosphoramidite, phosphine or any other phosphorus (III) derivative as part of the synthesis of DNA, RNA, phosphoropeptides, phosphonopeptides, phosphorylated nucleoside sugars, or oligosaccharides or any other thiolated phosphorus (V) derivatives prepared either in solution or in the solid-phase.

The starting phosphorus-containing compound, and its corresponding phosphorothioate produced by the method of the present invention, have the formulae B and C, respectively:

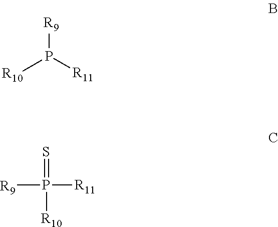

In the above formulae, R₉, R₁₀, and R₁₁ may be the same or different and are preferably selected from organic moieties such as optionally substituted alkyl, alkoxy, phenyl, phenoxy, and tertiary amino, and analogues of the foregoing.

Preferably, R₉ and R₁₀ are independently selected from the group consisting of —R₁₂, —OR_{E}, —C(R₁₄)(R₁₅)(R₁₆), —NH(R₁₇), —N(R₁₈)(R₁₉) or —S—R₂₀; R₁₁ is selected from the group consisting of —R₁₂, —OR_{E}, —C(R₁₄)(R₁₅)(R₁₆), —NH(R₁₇), —N(R₁₈)(R₁₉) or —S—R₂₀, halogen, or a protecting group. Each R₉, R₁₀, and R₁₁ may be the same or different. Also, each R group (i.e., R₁₂-R₂₀) may be the same or different, and are preferably selected from the group consisting of aryl groups, alkyl groups, alicyclic groups, carbohydrate groups, glyceride groups, peptide groups, fluorophores, nucleoside groups, amino acid groups, steroidal groups, terpene groups, oligonucleotide groups, phosphonopeptide groups, phospholipid groups, phosphorus-containing polymeric groups, and phosphonate-modified surfaces. Preferably, the R groups in Formulae (B) and (C) are alkyl groups (preferably (C₁-C₈)alkyl groups), peptide groups, and/or oligonucleotide groups. More preferably, the R groups of Formula (B) are peptide and oligonucleotide groups.

Referring to Formula (B), the term “carbohydrate groups” means inositol, polyhydroxy aldehyde groups, polyhydroxy ketone groups and other groups that can be hydrolyzed to the same. Monosaccharidic, disaccharidic, and polysaccharidic groups that may or may not carry specific hydroxy protecting groups, are included within the scope of this term. The term “glyceride group” means glycerol groups that may or may not carry specific hydroxy protecting groups. The term “peptide group” means amide-containing groups formed by the interaction between amino groups and carboxyl groups of amino acids. This term encompasses dipeptides, tripeptides, and polypeptides up to a molecular weight of 10,000, that may or may not carry specific hydroxy protecting groups.

The term “nucleoside group” is one that is formed from a sugar (notably ribose or deoxyribose) with a purine or pyrimidine base by way of an N-glycosyl link. This includes, but is not limited to adenosine, cytidine, guanosine, uridine, thymidine, inosine, their 2′-deoxy and 2′-substituted analogues, synthetic derivatives and the like that may or may not carry specific hydroxy protecting groups. The term “amino acid group” means amino acid groups such as alanine, valine, glutamine, lycine, histidine, isoleucine, proline groups, and the like that may or may not carry specific hydroxy protecting groups. The term “steroidal groups” means groups containing a tetracyclyl cyclopenta[a]phenanthrene skeleton, such as aldosterone, androsterone, cholecalciferol, cholesterol, choleic acid, corticosterone, cortisol, cortisol acetate, cortisone, cortisone acetate, deoxycorticosterone, dexamethasone, ergocalciferol, ergosterol, estradiol-17.alpha., estradiol-17.beta., estriol, estrone, lanosterol, lithocholic acid, progesterone, testosterone, and the like that may or may not carry specific hydroxy protecting groups. The term “terpene group” means groups of (unsaturated) hydrocarbons having the formula C₁₀H₁₆, which are based upon the isoprene unit C₅H₈, which may be acyclics or cyclic with one or more benzenoid groups. This includes dipentene, pinene, mysene, menthane groups, and the like that may or may not carry specific hydroxy protecting groups.

The term “oligonucleotide group” means a group typically containing 2-1000 nucleotides, and even larger polynucleotides. The term “nucleotide” means any compounds containing a heterocyclic compound bound to a phosphorylated sugar by an N-glycosyl link. Exemplary of such compounds are adenosine phosphate, flavin mononucleotide, and the like; but more specifically, the term also encompasses molecules which are combinations of a nucleic acid purine or pyrimidine, one sugar (usually (a chemically modified) ribose or deoxyribose), and a phosphate group, exemplary of such nucleotides would be adenylic acid, guanylic acid, uridylic acid, cytidylic acid, and the like that may or may not carry specific protecting groups. The term “nucleotide” furthermore encompasses morpholino-oligonucleotides, where 6-membered morpholine rings replace ribose or deoxyribose rings and in which nucleotides are linked through P^{V} centered moieties.

The term “phosphonopeptide” means a phosphorus-containing peptide derivative in which the carboxamide linkage between the two amino acids is replaced by a phosphono group.

The term “phospholipid” means a bifunctional, trifunctional or multifunctional unit having a phosphorus group attached to one function and one or more long chain organic (>C₆) groups at the other functions and that may or may not include protecting groups. The term “phosphorus-containing polymer” means oligomers of greater than 5 units which contain phosphorus in the backbone or on the periphery of the backbone. The term “phosphonate-modified surface” means a glass, metal silicon, inorganic substrate, or other support having a phosphonate layer or multilayers with or without metals or other substrates in the layer or layers. In one embodiment, the organophosphate compound is a flame-retarding phosphonopeptide or phosphorous-containing polymer.

The trivalent phosphorus functional groups of the phosphorus-containing compounds can be selectively sulfurized. Alternatively, all of the trivalent phosphorus groups can be sulfurized. Furthermore, more than one phosphorus functional group can be sulfurized at a time, or they can be sulfurized sequentially (i.e., one at a time in a stepwise manner). For example, in the sulfurization of oligonucleotides it is desirable to sulfurize one nucleotide at a time. This prevents cleavage of the oligonucleotides when hydroxy protecting groups are removed by acidolysis.

For oligonucleotides, or other similar molecules, the sulfur-transfer reagents of Formula (A) do not modify the nucleosidic residues, thereby preserving the chemical identity of the macromolecule. Thus, the reagents of Formula (A) and the method of the present invention can be reliably used in the automated synthesis of desired compounds.

In one embodiment, R₉ and R₁₀ may be ribonucleosides and deoxyribonucleosides and synthetic analogues thereof. The reagents of the present invention are particularly useful in the synthesis of phosphorothioate analogs of oligonucleotides from a phosphite or phosphonous ester in which R₉ and R₁₀ are nucleosides, particularly suitably protected nucleosides. This has particular application in (solid-phase) oligonucleotide synthesis, to produce internucleotide phosphorothioate or phosphonothioate bonds in a nucleotide multimer. As addressed in the background description, sulfurization is a common step in the synthesis of oligonucleotides containing one or more P═S bonds.

As the phosphorothioate moiety obtained by the present invention is less susceptible to (enzymatic) hydrolysis, it is preferred to incorporate the sulfurization reagent of the invention in an oligonucleotide to improve stability.

“Oligonucleotide” includes, but is not limited to phosphodiesters, phosphotriesters, phosphorothioates, phosphodithioates, phosphorothiodiamidate and H-phosphonates derivatives. It encompasses also both naturally occurring and synthetic oligonucleotide derivatives.

Generally, after completion of the synthesis, the pentavalent P═S containing unit(s) are subjected to removal of one or more protecting groups on the phosphorus atom, thus creating a phosphorothioate diester of general chemical formula R₉—O—P(═S)(—O⁻)—O—R₁₀. In such case, R₁₁ is preferably a group which can be selectively removed (cleaved) following completion of the oligonucleotide. An example of such a group is β-cyanoethyl. However, if it is desired to produce a phosphonothioate analog of a nucleotide multimer (i.e., an analog in which at least one phosphonous linking group has an P═O replaced with P═S), then R₁₁ need not be a group which can be selectively removed following sulfurization. As addressed above, instead of phosphorodiester linkages it may be beneficial to use phosphorothiodiamidate intersubunit linkages instead, making use of morpholino nucleotide monomers instead of ribosyl or deoxyribosyl monomers for R₉ and R₁₀.

A person skilled in the art will recognize that there are many synthetic derivatives of oligonucleotides, and that use of compound (A) as a sulfurization reagent to introduce sulfur atoms in any synthetic derivatives of oligonucleotides are thus covered.

The sulfurization reaction occurs in one or more solvents. It is therefore, that the sulfurization reagents of structure (A) can be applied in combination with a variety of solvents, including, but not limited to, methanol, ethanol, 1-propanol, 2-propanol, ethylene glycol, propylene glycol, acetone, N,N-dimethylformamide, N,N-dimethylacetamide, acetonitrile, dimethylsulfoxide, pyridine, picoline, lutidine, collidine, toluene, xylene, benzene, diethyl ether, hexane, heptane, pentane, petroleum ether, methyl tert-butylether, N-methylpyrrolidone, chloroform, dichloromethane, ethyl acetate, methyl acetate, acetic anhydride, acetic acid, trifluoroacetic acid, dichloroacetic acid, trichloroacetic acid, chloroacetic acid, 1,2-dichloroethane, 1,1-dichloroethane, 1,1,1-trichloroethane, tetrahydrofuran, furan, tetrahydropyran, 1-butanol, 2-butanol, s-butanol, tent-butanol, trifluoroethanol, hexafluoroisopropanol, formamide, triethylamine, N,N-diisopropylethylamine, cyclohexane, dioxane, piperidine, and any combination thereof. Preferably, the solvent(s) is (are) one in which both the reagent and the compound subject to sulfurization are sufficiently soluble, and, in the case of solid-supported synthesis, is (are) also compatible with the resin to allow the soluble compound (A) to access the on-resin phosphorus functionality, such that the reaction occurs in a reasonable amount of time. In a preferred embodiment, the reaction takes place in a combination of dimethylsulfoxide and a pyridine. In a more preferred embodiment, the pyridine is 3-picoline.

In sulfurization reactions, either in solution or solid phase, the reaction mixture can contain, besides the sulfurization reagent(s) and one or more solvents, a base as co-reagent. In some cases, this base can be (one of) the solvents. The base is preferably an organic nitrogen base. The volume ratio of sulfurization agent and base is preferably in the range of 3:1 to 1:3, more preferably 2:1 to 1:2. The bases include, but are not limited to, (substituted) pyridine (e.g. picoline, lutidine, collidine), NH₃, ammonium hydroxide, hydroxylamine, (substituted) alkylamine (i.e. triethylamine, N,N-diisopropylethylamine, benzylamine) or (substituted) heterocyclic amine (e.g. piperidine, morpholine, triazole, tetrazole). In a preferred embodiment, the base is a pyridine. In a more preferred embodiment, the pyridine is 3-picoline.

In sulfurization reactions, either in solution or solid phase, the reaction mixture can contain, besides the sulfurization reagent(s), solvent(s) and an optional base, an anionic sulfur salt (i.e. containing S²⁻ or RS⁻). These sulfide salts include, but are not limited to, sulfide, disulfide, trisulfide, tetrasulfide salts of sodium, potassium, lithium, magnesium and/or calcium. These are commonly included to accelerate the reaction and/or reduce the amount of reagent equivalents.

Preferably, the reaction occurs in less than about 1 hour, more preferably in less than about 30 minutes, most preferably in less than about 15 minutes. In some applications, the reaction can be complete in as little as 30 seconds. Although it is desirable to carry out the reaction at room temperature (i.e., about 25-30° C.), it can be carried out at a temperature within a range of about 0-50° C., and preferably about 10-50° C. Typically, the conversion of a compound of Formula (B) to the thioated derivative of Formula (C) is greater than about 90%, and frequently greater than about 99%.

Here above, sulfurization is discussed for oxidation of P^{III} to pentavalent P^{V}. In analogy, sulfurization is also applicable to oxidation of olefins that—in sulfurized form—can be used as additives for lubricants. Reference is made to U.S. Pat. No. 5,403,960, and references cited therein. Especially suitable starting olefins for use in the present invention are the monoethylenically unsaturated aliphatic hydrocarbons referred to as aliphatic monoolefins containing 3 to about 6 carbon atoms. These include 1-butene, 2-butene, isobutene, 1-pentene, 2-pentene, 2-methyl-1-butene, 3-methyl-1-butene, 2-methyl-2-butene, 1-hexene, 2-hexene, 3-hexene, 2-methyl-1-pentene, 2-methyl-2-pentene, 2-ethyl-2-butene and the like, including mixtures thereof.

Preferably, the olefins are branched chain olefins such as isobutene, 2-methyl-1-butene, 1-methyl-2-butene, 2-methyl-2-pentene and the like. More preferably, the ethylenically double bond adjoins a tertiary carbon atom such as isobutylene, the most preferred olefin.

It is immediately clear to the skilled person how to conduct the reaction of such an olefin, either as a gas or a liquid, with the sulfurization agent of the present invention.

### EXAMPLES

### Example 1

### Preparative Sulfurization of Triphenyl Phosphite

To 25 μL of a solution of a 1 M triphenyl phosphite in MeCN was added 500 μl, of a 0.15 M solution of FMDS in DMSO/3-picoline (1/1, v/v) and the solution was allowed to stand (without stirring) for 16 h. It is noted that 3-picoline is a base, thus creating basic conditions. The mixture was extracted with large volumes of water and dichloromethane. After drying of the organic layer on sodium sulfate, the solvents were removed in vacuo. After silica gel column chromatography, all collected fractions were analyzed for ³¹P content. The only signal on ³¹P NMR (MeCN-d₃) to be found was at δ 54.2 ppm, corresponding to the literature value of (PhO)₃P═S (Nucleos. Nucleot. Nucl. Acids 2005, 24, 1293). In contrast, no oxidized product (PhO)₃P═O (δ invullen, δ˜0 ppm) nor starting material (PhO)₃P (δ 128.3 ppm) were found in any of the fractions. Hence, FMDS was found successful in basic conditions. Besides, it should be noted that commercially available FMDS as mentioned above is in its stable dihydrochloride (acidified) form. Said otherwise, FMDS showed pH independent stability.

### Comparative Example I

### Comparison of PADS and FMDS

To assay the efficiency of FMDS, a panel of reactions were compared to the sulfurizing reagent PADS under identical conditions. Thus, whereas the PADS reaction (25 μL of a 1 M solution of (PhO)₃P in MeCN added to a solution of 0.2 M of slightly aged (3 d old) PADS in MeCN/3-picoline 1/1 (v/v) containing 200 eq. PADS) resulted, after overnight standing (not shaking nor stirring) in a mixture containing (as judged by ³¹P NMR—see example 1) at least 35% unreacted starting material. By contrast, reactions with all FMDS combinations (100/50/10 eq. of 0.15 M FMDS in DMSO/3-picoline 1/1 (v/v) overnight or 50 eq. FMDS 4 h before NMR analysis) showed complete conversion of the starting material.

### Example 2

### Sulfurization of a 3′-Phosphitetriester Linked Nucleotide to Phosphorothioate

The effect of FMDS on sulfurization of a nucleotide 3′-phospitetriester was determined in the following reaction. Commercially available 2′-O-methyl adenosine nucleotide N,N-diisopropyl-2-O-cyanoethyl phosphoramidite (5′-O-DMT, base protected with Bz), 50 μmol as a 0.1 M solution in MeCN was added, in an argon atmosphere, to a mixture of 6-chlorohexanol (8 eq.) and 1H-tetrazole (3 eq., as a 0.45 M solution in MeCN) in dry 1,2-dichloroethane (2 mL) containing activated molecular sieves (4 Å). After 2 h of reaction, ³¹P NMR (DMSO-d₆) showed conversion of starting material (δ 150.2 & 149.9 ppm, diastereomers) to the phosphitetriester (δ 139.9 & 139.4 ppm, diastereomers). FMDS (0.15 M in DMSO/3-picoline 1/1 (v/v), 12.5 eq.) was added and the mixture was analysed by ³¹P NMR after 1 h. The presence of P^{III} (compared to P^{V} at δ 68.0 & 67.6 ppm, diastereomers) was <4.5%, whereas no P═O was observed.

It should be noted that these conditions employ smaller amounts of sulfurization reagent than are commonly used in solid-phase oligonucleotide synthesis (>100 eq. in the prior art, compared to 12.5 eq. here).

1.-13. (canceled)

14. A method of sulfurizing a compound comprising:
reacting said compound with a sulfurizing agent of formula A: 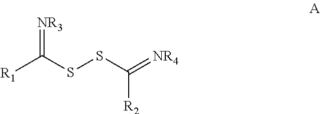
or a salt, hydrate, solvate, or a mixture thereof, wherein:
R₁, R₂, R₃ and R₄ are each independently selected from the group consisting of H, —OH, —NO₂, —CN, —SO₂Ra, —NHRa, —N(Ra)₂, —C(O)Ra, —CO₂Ra, —ORa, halogen, alkyl, alkenyl, alkynyl, aryl, aralkyl, and alkoxyl, and
Ra, independently for each occurrence, is selected from the group consisting of H, halogen, alkyl, aryl and aralkyl, optionally containing one or more heteroatoms selected from the group consisting of O, N, P, Se, B and S.

15. The method of claim 14, wherein,
the sulfurizing agent is represented by formula A1: 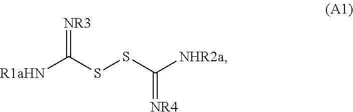
R₃ and R₄ are nitrogen-containing moieties, and
R₁ₐ and R₂ₐ are each independently selected from the group consisting of H, halogen, alkyl, aryl and aralkyl, optionally containing one or more heteroatoms selected from the group consisting of O, N, P, Se, B and S.

16. The method according to claim 14, wherein R₁ and R₂ are nitrogen-containing moieties.

17. The method according to claim 16, wherein R₁ and R₂ are NH₂.

18. The method according to claim 14, wherein said sulfurizing agent is formamidine disulfide.

19. The method according to claim 14, wherein said compound contains trivalent phosphorus P^{III}.

20. The method according to claim 19, wherein said compound containing trivalent phosphorus P^{III} is an oligonucleotide, or a derivative thereof.

21. The method according to claim 19, wherein said compound containing trivalent phosphorus P^{III} is converted to a flame-retarding organophosphate compound having P^{V} by said reaction.

22. The method according to claim 14, wherein said compound is an olefinic compound.

23. The method according to claim 14, wherein said reacting further involves a base as a co-reagent.

24. The method according to claim 23, wherein said base is a (substituted) pyridine.

25. The method according to claim 24, wherein said base is pyridine or 3-picoline.

26. The method according to claim 14, wherein said reacting further involves at least one sulfide salt.

27. The method according to claim 14, where said at least one sulfide salt is sodium sulfide.
